(19) United States
(12) Patent Application Publication
(10) Pub. No.: US 2025/0318718 A1
(43) Pub. Date: Oct. 16, 2025

(54) SYSTEMS, DEVICES, AND METHODS FOR DELIVERY OF MEDICAL DEVICES

(71) Applicant: Boston Scientific Scimed, Inc., Maple Grove, MN (US)

(72) Inventors: Ryan V. WALES, Shrewsbury, MA (US); Andrew James SCHAUBHUT, Bolton, MA (US); Kensuke HAYASHI, Penang (MY)

(73) Assignee: Boston Scientific Scimed, Inc., Maple Grove, MN (US)

(21) Appl. No.: 19/172,918

(22) Filed: Apr. 8, 2025

Related U.S. Application Data
(60) Provisional application No. 63/632,101, filed on Apr. 10, 2024.

(51) Int. Cl. A61B 1/018; A61B 1/00; A61B 17/00; A61B 17/02

(52) U.S. Cl. CPC A61B 1/018; A61B 1/00082; A61B 17/0218; A61B 2017/00269

(57) ABSTRACT

A medical device may comprise a first wall defining a first lumen. The first lumen may be configured to receive a shaft of a medical scope. Further, the medical device may include at least one second wall defining a second lumen. The at least one second lumen may be configured to receive an accessory device. The at least one second wall may include a perforation that may extend longitudinally along the at least one second wall.

### CROSS-REFERENCE TO RELATED APPLICATION(S)

This application claims the benefit of priority to U.S. Provisional Application No. 63/632,101, filed on Apr. 10, 2024, which is incorporated by reference herein in its entirety.

### TECHNICAL FIELD

Various aspects of this disclosure relate generally to systems, devices, and methods for delivery of medical devices. Aspects of the disclosure relate to systems, devices, and methods for delivering traction devices in order to apply traction to a tissue to, for example, facilitate resection of the tissue.

### BACKGROUND

Medical devices, such as endoscopes or other suitable insertion devices, are employed for various diagnostic and surgical procedures, such as endoscopy, laparoscopy, arthroscopy, etc. An insertion device may be inserted into a subject's body through a surgical incision or via a natural anatomical orifice to perform a procedure. For example, endoscopic submucosal dissection (ESD) removes cancerous or other lesions from the gastrointestinal tract. In some procedures, such as ESD, accessory devices may be utilized along with the insertion device. A need exists for systems, devices, and methods for delivery of accessory devices.

### SUMMARY

Aspects of the disclosure relate to, among other things, systems, devices, and methods for delivery of medical devices. Each of the aspects disclosed herein may include one or more of the features described in connection with any of the other disclosed aspects.

In an example, a medical device may comprise a first wall defining a first lumen. The first lumen may be configured to receive a shaft of a medical scope. Further, the medical device may include at least one second wall defining a second lumen. The at least one second lumen may be configured to receive an accessory device. The at least one second wall may include a perforation that may extend longitudinally along the at least one second wall.

According to some aspects, the medical device may comprise a plurality of second walls defining a plurality of second lumens. The plurality of second lumens may be positioned circumferentially around the first lumen. The plurality of second walls may include a perforation extending longitudinally along a respective second wall of the plurality of second walls. The plurality of second lumens may be positioned circumferentially around the first lumen. The perforation may be on an outer surface of the respective second wall. The perforation may extend from a distalmost end of the respective second wall to a proximalmost end of the respective second wall. The perforation may be a second perforation, and the first wall may include a first perforation. The first perforation may extend longitudinally along the first wall. The first perforation may extend from a distalmost end of the first wall to a proximalmost end of the first wall. Furthermore, the medical device may be a sleeve that may be configured to extend from a distal tip of the shaft to a proximal portion of the shaft that remains external to a body lumen.

In addition, the medical device may include a tab may be formed adjacent to a proximal end of the at least one second wall. A second tab may also be formed adjacent to a proximal end of the first wall. A proximal end of the first wall may have a recessed or notched portion. The at least one second may have an open proximalmost end and an open distalmost end and the first lumen may have an open proximalmost end and an open distalmost end.

The disclosure may also include a medical device comprising a membrane defining a central lumen configured to receive a scope device and a plurality of outer lumens. The plurality of outer lumens may be configured to receive an accessory device, and a perforation may extend longitudinally along a wall defining at least one of the plurality of outer lumens.

Any of the devices disclosed herein may include any of the following features. According to some aspects, the membrane may include a plurality of perforations. Each perforation of the plurality of perforations may extend longitudinally along a respective wall defining each of the plurality of outer lumens.

The disclosure may also include another medical device comprising an expandable member. The medical device may comprise a first lumen extending through the expandable member and that may be configured to receive a shaft of a scope device. A perforation may extend longitudinally along a wall defining at least one of the plurality of outer lumens. According to some aspects, the expandable member may include an absorbent member.

Any of the devices disclosed herein may include any of the following features. The medical device may further include a ring disposed within the first lumen. The expandable member may include a balloon.

Furthermore, any of the devices, methods, or systems disclosed herein may include any of the following features, alone or in any combination or subcombination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of an exemplary medical device according to aspects of this disclosure;

FIG. 2 is a perspective view of the distal tip of the medical device of FIG. 1 and a distal portion of a delivery device, according to aspects of this disclosure;

FIG. 3A is a perspective view of the distal tip of the medical device of FIG. 1 and a distal portion of an alternative delivery device, according to aspects of this disclosure;

FIG. 3B is a perspective view of a proximal portion of the medical device of FIG. 1 and the delivery device of FIG. 3A;

FIG. 4 is a perspective view of a proximal portion of the medical device of FIG. 1 and a proximal portion of another delivery device, according to aspects of this disclosure;

FIGS. 5A-5B are perspective views of another exemplary delivery device, according to aspects of this disclosure;

FIG. 5C is a cross-sectional view of the delivery device of FIGS. 5A-5B in use with the medical device of FIG. 1; and

FIG. 6 is a perspective of the medical device of FIG. 1 and another exemplary delivery device, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value. Unless otherwise stated, ranges disclosed herein include the end points of the ranges. The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element.

A medical device, such as an endoscope, colonoscope, or other type of scope device may have a working channel. The medical device may be navigated to a procedure site (e.g., a target site). Accessory instrument(s) may be inserted into a working channel of the medical device in order to perform aspects of a procedure. In some examples, it may be desirable to utilize more than one accessory device simultaneously during a procedure. For example, a user may desire to apply traction to a tissue with a first medical device while resecting the tissue with a second medical device. Aspects of the disclosure relate to delivery devices for facilitating use of multiple accessory devices. For example, disclosed are delivery devices that facilitate delivery of one or more accessory devices outside of a shaft of the medical device or medical scope (e.g., over-the-scope delivery of the accessory devices). For example, the delivery devices may define one or more lumens for facilitating over-the-scope (or outside of the scope) delivery of accessory devices.

In examples, accessing a procedure site may include endoluminal placement of the medical device into the patient, such as through an anatomical passageway via a natural orifice. The orifice can be, for example, the nose, mouth, or anus, and the placement can be in any portion of the GI tract, including the esophagus, stomach, duodenum, large intestine, or small intestine. Placement also can be in other organs or other bodily spaces reachable via the GI tract, other body lumens, or openings in the body. This disclosure is not limited to any particular medical procedure or treatment site within a body.

Examples of the disclosure may relate to devices and methods for performing various medical procedures and/or treating portions of the large intestine (colon), small intestine, cecum, esophagus, pancreatic-biliary tract, any other portion of the gastrointestinal tract, and/or any other suitable patient anatomy (collectively referred to herein as a “target treatment site”). As mentioned above, this disclosure is not limited to any specific medical device or method, and aspects of the disclosure may be used in connection with any suitable medical tool and/or medical method, at any suitable site within the body.

Reference will now be made in detail to aspects of the disclosure, examples of which are illustrated in the accompanying drawings. Wherever possible, the same or similar reference numbers will be used through the drawings to refer to the same or like parts.

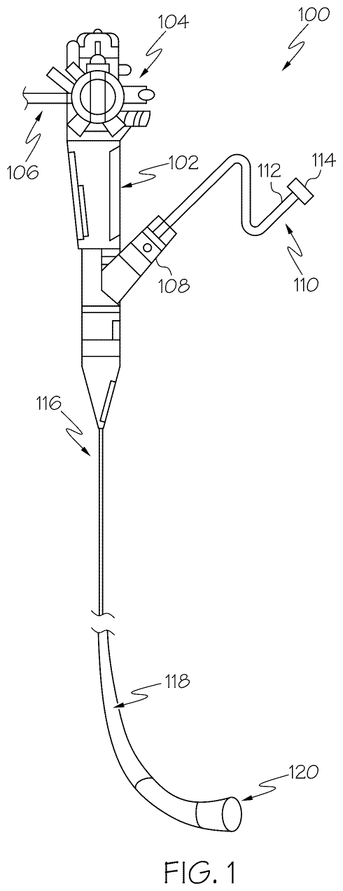

FIG. 1 depicts an exemplary medical device 100. Medical device 100 may include an insertion device, such as an endoscope, which may be inserted into a body lumen of a patient. Medical device 100 may include a handle 102 and a shaft 116 extending distally from handle 102. Shaft 116 may include one or more working channels extending from a port 108 of handle 102 to a distal tip 120 at a distal portion 118 of shaft 116.

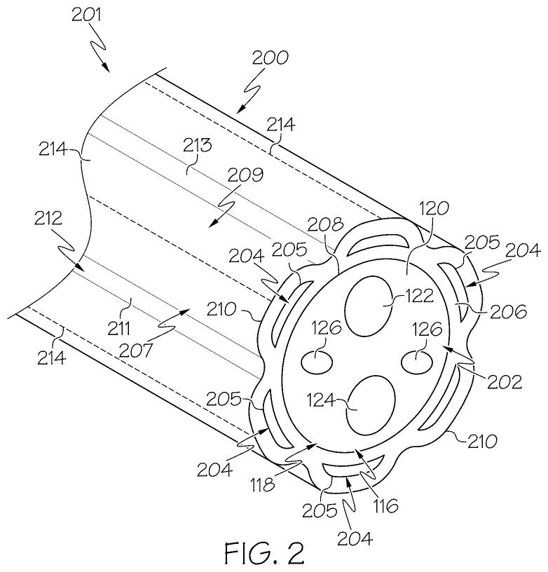

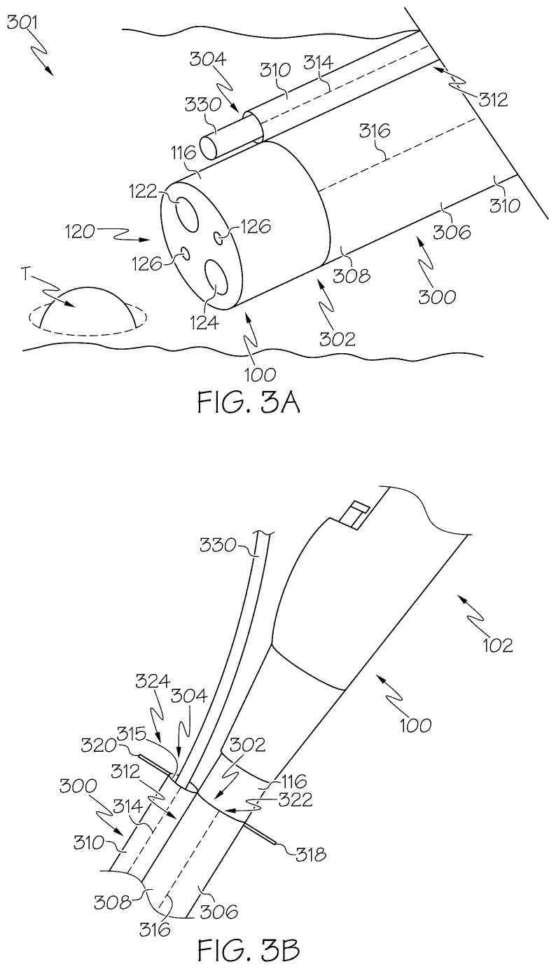

Distal tip 120 may be a distalmost portion of distal portion 118 of shaft 116. Distal tip 120 may include a distal opening 124 (FIGS. 2 and 3A) of the working channel. In some embodiments, distal tip 120 may include an imaging element 122 and/or one or more lighting elements 126 (all shown in FIGS. 2 and 3A) to aid in accurately positioning shaft 116 adjacent a procedure site during a procedure and to assist in performing the procedure (e.g., with one or more accessory devices). Although not shown, distal tip 120 may also include one or more openings for delivery of air, water, and/or suction.

A user of medical device 100 may insert a medical instrument 110 (e.g., an accessory device) into port 108 on handle 102. Medical instrument 110 may include a shaft 112 and a proximal end 113 that remains external to handle 102. Proximal end 114 may define a user interface (e.g., handle) of medical instrument 110 for access and control by a user of medical instrument 110. The shaft 112 may be advanced through the working channel until a distal portion (e.g., an end effector) of medical instrument 110 extends out of distal opening 124.

In some embodiments, medical device 100 may include a port 106, which may connect handle 102 to sources of, for example, air, water, suction, power, image processing, and/or viewing equipment. Handle 102 may include one or more actuators 104 at or along a proximal portion of handle 102 (e.g., adjacent to port 106), for example, to control the movement of shaft 116, and particularly distal portion 118, the activation of one or more imaging element(s) 122 and lighting element(s) 126, and control a deflection, position, or orientation of distal tip 120.

It should be appreciated that control of distal tip 120 may additionally be provided by moving handle 102, such as by pushing and/or pulling handle 102 to advance or retract distal tip 120 relative to the procedure site. Distal tip 120 of medical device 100 (e.g., an endoscope) may be “forward-facing” in that the features of distal tip 120 may face distally (i.e., forward of a distalmost face of distal tip 120).

Although medical device 100 is discussed above as being an endoscope, this disclosure is not so limited. It will be appreciated that, unless otherwise specified, duodenoscopes, endoscopes, gastroscopes, endoscopic ultrasonography (“EUS”) scopes, colonoscopes, ureteroscopes, bronchoscopes, laparoscopes, cytoscopes, aspiration scopes, sheaths, catheters, or any other suitable delivery device or insertion device may be used in connection with the systems, devices, elements, assemblies, methods, etc. described herein.

FIG. 2 illustrates a distal portion of a system 201, including medical device 100 and a sleeve 200 positioned around shaft 116 (e.g., distal tip 120 and/or distal portion 118). Sleeve 200 may radially surround at least a portion of the length of shaft 116 of medical device 100. Sleeve 200 is comprised of a membrane 206 defining a central lumen 202 and one or more auxiliary lumen(s) 204 disposed circumferentially around central lumen 202 and shaft 116.

As discussed in further detail below, sleeve 200 fits around an outer surface of shaft 116 such that a distal end of medical instrument 110 may extend distally out of distal opening 124 for accessing the procedure site when distal portion 118 is positioned at the procedure site. Auxiliary lumen(s) 204 (i.e., outer lumens) facilitate delivery of additional medical instruments when shaft 116 is introduced to a target or procedure site. Membrane 206 may be removed to decouple the additional medical instruments from the shaft 116, as discussed below.

Central lumen 202 may be sized and shaped to receive shaft 116 and distal tip 120 of shaft 116. Central lumen 202 may include open proximal and distal ends, so distal tip 120 may be inserted into the proximal end of central lumen 202. Distal tip 120 may be extended distally through central lumen 202 and extended through the distal opening of central lumen 202 in order to install sleeve 200 on shaft 116. A distalmost end of distal tip 120 may align with a distalmost end of central lumen 202 or may be distal to a distalmost end of central lumen 202. Alternatively, membrane 206 surrounding central lumen 202 may extend distally to a distalmost end of distal tip 120. In such embodiments, membrane 206 may be transparent or otherwise non-interfering with visualization using the imaging element 122, ensuring clear and unobstructed views during procedures.

Central lumen 202 may be defined by an inner wall 208 of the membrane 206. The shape of inner wall 208 may form the shape of central lumen 202. For example, inner wall 208 may be tubular, resulting in a tubular central lumen 202. The size of the inner wall 208 may also include a diameter/width sufficient for central lumen 202 to accommodate a corresponding diameter/width of shaft 116. In an embodiment, central lumen 202 may have a snug fit radially surrounding shaft 116. In some examples, central lumen 202 may have a diameter/width that allows shaft 116 to move longitudinally or rotationally within central lumen 202 relative to membrane 206.

In such examples, an inner surface of inner wall 208 may include coatings or textures to reduce friction and facilitate smooth movement of the endoscope within sleeve 200. Alternatively, inner wall 208 may have a friction fit with shaft 116, such that shaft 116 is not rotationally or longitudinally movable within central lumen 202 (i.e., shaft 116 may be fixed relative to inner wall 208). The materials described above may be flexible enough to conform to the shape of shaft 116 (e.g., a tubular shape) and accommodate movement of medical device 100 during procedures.

Auxiliary lumen(s) 204 may be circumferentially disposed around the central lumen 202 and shaft 116 to accommodate and guide additional medical instruments alongside shaft 116 of the device. Auxiliary lumen(s) 204 help the simultaneous delivery of multiple instruments, allowing medical device 100 to deliver additional medical instruments simultaneously.

Auxiliary lumen(s) 204 may be positioned radially outwardly relative to central lumen 202, around central lumen 202. Auxiliary lumen(s) 204 serve as conduits for accessory devices to be inserted alongside and outside of shaft 116 of medical device 100. For example, auxiliary lumen(s) 204 may be positioned circumferentially around either the entire circumference or a portion of the circumference of central lumen 202. Sleeve 200 may serve to facilitate coupling the accessory devices to the shaft 116.

Accessory devices within auxiliary lumen(s) 204 may be coupled around the circumference of shaft 116 based on user preferences and procedural requirements. The number and size of auxiliary lumen(s) 204 (e.g., six auxiliary lumens 204 in FIG. 2) are merely exemplary. For example, sleeve 200 may include more auxiliary lumen(s) 204 if additional accessory devices are needed (e.g., eight auxiliary lumen(s) 204.) Additionally, auxiliary lumen(s) 204 may be sized, shaped, and/or otherwise configured to receive various accessory devices. The size of the diameter of auxiliary lumen(s) 204 may be approximately 2.0 mm to 3.8 mm or approximately 2.8 mm to 3.2 mm. Alternatively, auxiliary lumen(s) 204 may have any other suitable size to accommodate accessory devices that may include but are not limited to graspers, forceps, clips, snares, hooks, needles, or any other suitable device.

Auxiliary lumen(s) 204 include openings at both proximalmost and distalmost ends 205 of auxiliary lumen(s) 204, providing entry and exit points for accessory devices to be introduced into respective auxiliary lumen(s) 204. In other words, the auxiliary lumen(s) or second lumens (and central lumen 202) has an open proximalmost end and an open distalmost end. For example, proximalmost openings allow for the insertion of the distal end of an accessory device into an auxiliary lumen(s) 204, while the distalmost opening of auxiliary lumen(s) 204 enables the extension of the accessory device distally out of auxiliary lumen(s) 204. Upon reaching the distal opening of distalmost end 205, the accessory device extends out of auxiliary lumen(s) 204.

Outer walls 210 of membrane 206 may define radially outer boundaries of auxiliary lumen(s) 204 and inner wall 208 of membrane 206 may define radially inner boundaries of auxiliary lumen(s) 204. Outer walls 210 of membrane 206 may converge/intersect with inner wall 208 to form the boundaries of the auxiliary lumen(s) 204 at junctions 212. For example, a first lateral side 207 of one of outer walls 210 may have a first junction 211 (one of junctions 212) with inner wall 208. A second lateral side 209 of outer wall 210 may have a second junction 213 (another of junctions 212) with inner wall 208. Junctions 212 may extend approximately parallel to the central longitudinal axis of the sleeve 200 and/or shaft 116, extending from distalmost end(s) 205 of auxiliary lumen(s) 204 to proximalmost ends of the auxiliary lumen(s) 204. Junctions 212 may, therefore, span the entire length of auxiliary lumen(s) 204.

Still referring to FIG. 2, outer walls 210 may include perforations 214, which may be on an outer surface of outer walls 210. Although not shown in FIG. 2, inner wall 208 may also include one or more perforations to allow removal of membrane 206 from shaft 116. Perforations 214 allow for controlled tearing or removal of membrane 206, enabling instruments or devices inserted through auxiliary lumen(s) 204 to be uncoupled from sleeve 200 and shaft 116. Perforations 214 may extend longitudinally (e.g., approximately parallel to a central longitudinal axis of sleeve 200/shaft 116) along the length of auxiliary lumen(s) 204. Perforations 214 may extend partially or fully along the length of auxiliary lumens 204. For example, perforations 214 may extend the entire length of outer walls 210 of membrane 206 defining auxiliary lumen(s) 204 (from a distalmost end of outer wall 210 to a proximalmost end of outer wall 210). Alternatively, perforations 214 may also extend partially or on a portion of outer wall 210, for example, perforations 214 may be concentrated on the distalmost portion of sleeve 200.

Perforations 214 may be a series of openings or portions of reduced thickness of membrane 206. The shape of perforations 214 may be narrow and elongated, creating weakened areas along outer wall 210 that define auxiliary lumen(s) 204. Perforations 214 may be positioned in the same direction (e.g., parallel to a central longitudinal axis of sleeve 200) to facilitate tearing in a single direction. Depending on the requirements of the procedure or user preference, perforations 214 may vary in size, spacing, and distribution along the length of the auxiliary lumen(s) 204. In some examples, perforations 214 may be partially or fully torn to allow a user to choose between partial or full removal of membrane 206 based on the requirements of the procedure. Partial removal may be preferred when only a portion of shaft 116 or a portion of inserted instruments within auxiliary lumen(s) 204 need to be exposed/uncoupled, while complete removal may expose/uncouple the entire length of shaft 116 (e.g., any area previously covered by membrane 206).

Perforations 214 may allow for accessory devices to be removable from sleeve 200. For example, when a user reaches a target tissue site, the user may tear or otherwise open membrane 206 by pulling membrane 206 perpendicularly (or any angle causing perforations 214 to tear). The applied force causes membrane 206 to tear/open along perforations 214, resulting in decoupling accessory devices from sleeve 200 that extended through auxiliary lumen(s) 204 from shaft 116.

Each of sleeve 200, membrane 206, inner wall 208, and outer walls 210 may comprise suitable biocompatible materials such as silicone, thermoplastic elastomer, ethylene-propylene, fluorocarbon, or other similar materials. The material(s) may provide sufficient flexibility to navigate through patient anatomy and allow for a perforation 214. Additionally, inner walls 208 and outer walls 210 may be thin enough to allow for the formation of perforations 214 and for the inner walls 208 and outer walls 210 to be torn away. Inner walls 208 and outer walls 210 may be integrally formed of a single piece of material (e.g., extrusion) or may be separately formed.

Referring now to FIGS. 3A-3B, another exemplary system 301 including a sleeve 300 and medical device 100 is depicted. Sleeve 300 may include a membrane 306. Sleeve 300 may be similar to sleeve 200 except for the differences explicitly described herein. Accordingly, reference numerals adding 100 to the reference numbers used to describe system 201 are used to identify substantially similar components. Sleeve 200 may also have any of the properties of sleeve 300. Referring to FIG. 3A, sleeve 300 may surround at least a portion of the length of shaft 116 of medical device 100. Sleeve 300 may be comprised of a membrane 306 with a central (e.g., primary) lumen 302 and one or more auxiliary lumens 304 disposed on a radially outer side of central lumen 302.

Central lumen 302 may be sized and shaped to accommodate shaft 116 and serve as a passage through which shaft 116 and distal tip 120 may extend. Central lumen 302 may be defined by an inner wall 308 of membrane 306. Inner wall 308 of membrane 306 may have any of the properties of inner wall 208. Inner wall 308 defining central lumen 302 may include a perforation 316. Perforation 316 may have any of the properties of perforations 214 on outer walls of membrane 206 of sleeve 200.

Outer wall 310 of membrane 306 at least partially defines an auxiliary lumen 304, positioned radially (or laterally) outward relative to a central longitudinal axis of shaft 116 and central lumen 302. Auxiliary lumen may also be partially defined by inner wall 308. Auxiliary lumen 304 may have any of the properties of auxiliary lumen 204. Although FIGS. 3A-3B show a single auxiliary lumen 304, sleeve 300 may have a plurality of auxiliary lumens 304.

An accessory device 330 may extend through auxiliary lumen 304. Accessory device 330 may include, for example, a grasper, a forceps, a clip, a knife, a hook, a snare, a needle, a stapler, or any other suitable device (e.g., any accessory medical device). Outer wall 310 may join inner wall 308 at junction(s) 312. Junctions 312 may be similar to junctions 212. Junction(s) 312 may also extend longitudinally, approximately parallel to a central longitudinal axis of sleeve 300 and/or shaft 116. Additionally, outer wall 310 may include a perforation 314, which may have any of the properties of perforations 214.

FIG. 3B shows a proximal portion of shaft 116, accessory device 330, and sleeve 300, including a proximal end 322 of inner wall 308 and proximal end 324 of outer wall 310. A tab 320 may be formed adjacent to a proximal end or proximal opening 315 of auxiliary lumen 304. Tab 320 may have any shape that allows a user to hold or pinch tab 320 (e.g., elongated, rectangular, etc.). Tab 320 may be integrally formed with outer wall 310 or may be formed of a separate member from outer wall 310. Additionally, tab 320 may form a proximal portion of perforation 314 or be coupled to a portion of outer wall 310 adjacent to perforation 314.

A user may pull tab 320, for example, in a distal direction and/or transverse to a longitudinal axis of sleeve 300, such that perforations 314 split and outer walls 310 defining auxiliary lumen 304 separate from accessory device 330. Additionally, or alternatively, a user may pull accessory device 330 transversely (e.g., approximately perpendicularly outward) through perforation 314. Perforation 314 may first split at a proximal end 324 (FIG. 3B) of outer wall 310. The split or tear may extend distally and, in some examples, an entire length of outer wall 310 may split open at perforation 314. As a result, accessory device 330 may be separated from membrane 306. Subsequently, the user may independently navigate and actuate accessory device 330 and/or medical device 100. For example, medical device 100 may be moved to a retroflexed position while accessory device 330 is uncoupled from medical device 100.

In an embodiment, sleeve 300 may also include a pull tab 318 (e.g., similar to tab 320.) Pull tab 318 may be configured similarly to pull tab 320, except pull tab 318 may be configured to tear perforations 316 and inner walls 308 defining central lumen 302. For example, pull tab 318 may be pulled similarly to pull tab 320, but configured only to tear perforations 316, while perforations 314 remain intact. In other words, sleeve 300 may enable users to tear the membrane 306 along respective perforations (e.g., 314 or 316) by pulling one of pull tab 318 or pull tab 320. The presence of pull tab 318 and pull tab 320 may allow for controlled separation of membrane 306 to expose/uncouple either the accessory device 330 of the auxiliary lumen 304 or shaft 116 and distal tip 120 enclosed by central lumen 302 while ensuring that the other perforation (e.g., the other of 314 or 316) remains intact.

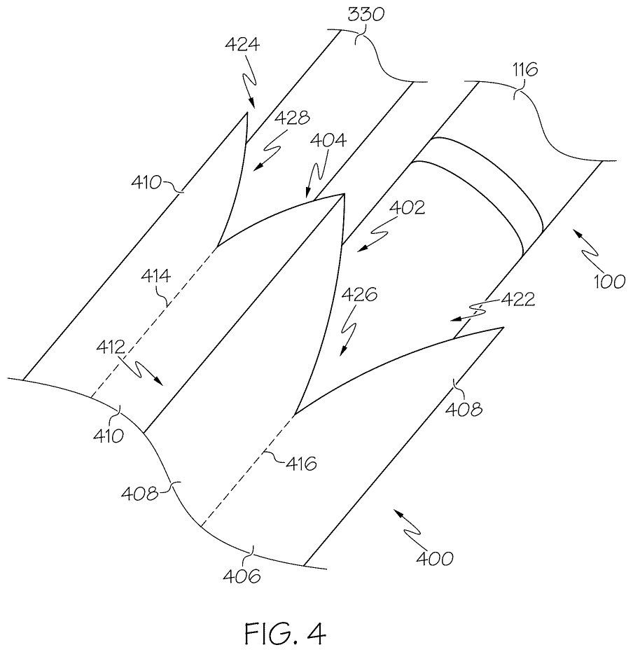

Referring now to FIG. 4, a sleeve 400 may have any of the properties of sleeves 200, 300 unless otherwise specified herein. FIG. 4 depicts a proximal portion of sleeve 400. Sleeves 200, 300 may also have any of the properties of sleeve 400. As discussed above for sleeves 200 and 300, sleeve 400 may surround at least a portion of the length of shaft 116. Where feasible, similar structures are identified by reference numerals that add 200 to the reference numerals used in FIGS. 2 and 100 to the reference numbers used in FIGS. 3A and 3B.

Sleeve 400 may have a membrane 406 that may have any of the properties of membrane 206 and membrane 306. Membrane 406 may have an inner wall 408 and an outer wall 410. Central lumen 402 (e.g., a primary lumen) may be defined by inner wall 408 (e.g., similar to inner wall 208 and outer wall 308) of the membrane 406. The shape of inner wall 408 may form the shape of central lumen 402. For example, inner wall 408 may be tubular, resulting in a tubular central lumen 402.

Outer wall 410 may define an auxiliary lumen 404 (e.g., similar to auxiliary lumens 204, 304). Outer walls 410 of membrane 406 may define radially outer boundaries of auxiliary lumen 404, and inner wall 408 of membrane 406 may define radially inner boundaries of auxiliary lumen 404. Outer walls 410 of the membrane may converge/intersect with inner wall 408 to form the boundaries of the auxiliary lumen(s) 404 at junctions 412 having any of the properties of junctions 212 and junctions 312. The number and size of auxiliary lumen 404 (e.g., one auxiliary lumen 404 in FIG. 4) are merely exemplary. For example, sleeve 400 may include additional auxiliary lumen(s) 404 (e.g., any number of auxiliary lumens 404, such as six distinct auxiliary lumen(s) 404.)

Still referring to FIG. 4, outer walls 410 may include perforations 414 having any of the properties of perforations 214 and perforations 314. Inner wall 408 may have a perforation 416, having any of the properties of perforation 316.

Perforations 414 and perforations 416 may extend partially or fully. For example, perforations 414 may extend the entire length of outer walls 410 of membrane 406, defining auxiliary lumen 404. In an embodiment, a proximal end 422 of inner wall 408 and a proximal end 424 of an outer wall 410 may have a recessed or notched portion 426 and a recessed or notched portion 428, respectively for helping to tear open perforations 414 and perforations 416. Recessed portion 426 and recessed portion 428 may have a concave shape formed by two curved sides converging to a central point.

A proximal portion (e.g., point) of recessed portion 426 may be aligned with perforation 416 and a proximal portion (e.g., point) of recessed portion 428 may be aligned with perforation 414. A user may tear open perforations 414 by moving accessory device 330 transversely (e.g., perpendicularly) to a central longitudinal axis of sleeve 400. Alternatively, a user may pull outer wall 410 transversely (e.g., at any angle causing perforations 414 to tear, such as an angle approximately perpendicular to a central longitudinal axis of sleeve 400) to separate accessory device 330 from shaft 116. Similarly, a user may tear open perforations 416 by moving shaft 116 transversely (e.g., perpendicularly) to a central longitudinal axis of sleeve 400. Alternatively, a user may pull inner wall 408 transversely (e.g., at any angle causing perforations 414 to tear, such as an angle that is approximately perpendicular to a central longitudinal axis of sleeve 400) to separate accessory shaft 116 from sleeve 400 and/or accessory device 330.

Recessed portions 426, 428 may help to, for example, orient accessory device 330 and/or shaft 116 so that accessory device 330 and/or shaft 116, respectively, may tear open perforations 414, 416, respectively. For example, recessed portion 426 may guide shaft 116 into a notched shape of recessed portion 426, such that shaft 116 may be moved perpendicularly to perforations 414. Similarly, recessed portion 428 may guide accessory device 330 into a notched shape of recessed portion 428, such that accessory device 330 may be moved perpendicularly to perforations 414.

Exemplary methods of using sleeves 200, 300, and 400 will be described. A user may insert distal tip 120 of shaft 116 into the proximalmost opening of the central (e.g., primary) lumen 202, 302, 402 of the sleeve 200, 300, 400. Subsequently, the user may advance shaft 116 through primary lumen 202, 302, 402 of sleeve 200, 300, 400. The user may advance shaft 116 through lumen 202, 302, 402 until distal tip 120 is at a desired position, for example, until aligned with a distalmost opening of lumen 202, 302, 402 or distal to a distalmost opening of lumen 202, 302, 402. The user may then insert accessory device 330 into auxiliary lumen 204, 304, 404. For instance, the user may insert a distal end of accessory device 330 into a proximalmost opening of lumen 204, 304, 404. After insertion, the user advances the accessory device through the auxiliary lumen 204, 304, 404 until the end effector of the accessory device extends out of the distalmost opening of the lumen 204, 304, 404. The user can repeat these steps for any number of accessory devices 330 and any number of auxiliary lumen(s) 204, 304, 404.

The user may insert shaft 116 of medical device 100, including sleeve 200 and accessory device(s) 330 into a body lumen, directing distal tip 120 toward a target tissue site. In some examples, the user may use accessory device(s) 330 to interact with tissue. For example, the user may use accessory device 330 to grasp or clip onto a target tissue, applying traction to a target tissue “T” (FIG. 3A). Then, the user may open perforations 214, 314, 414 or 216, 316, 416 using any of the methods/mechanisms described above.

Opening perforations 214, 314, 414 or 216, 316, 416 may uncouple, for example, accessory device 330 from shaft 116, or vice versa. In some examples, sleeve 200, 300, 400 may remain coupled to one or more of shaft 116 and/or accessory device 330. In other examples, sleeve 200, 300, 400 may be proximally removed from the body lumen. After shaft 116 and accessory device(s) 330 have been uncoupled from one another, the shaft 116 and accessory devices 330 may be separately navigated and/or actuated from one another. For example, accessory device(s) 330 may be anchored to tissue and shaft 116 may be moved (e.g., to a retroflexed position) to view accessory device(s) 330 and the target tissue “T.” One of accessory device(s) 330 that are or were positioned externally to shaft 116 and/or a medical instrument 110 inserted through the working channel of shaft 116 may be used to resect or otherwise treat the target tissue “T.” For example, a knife may be used to cut the target tissue “T” while the accessory device 330 applies traction to the target tissue “T.”

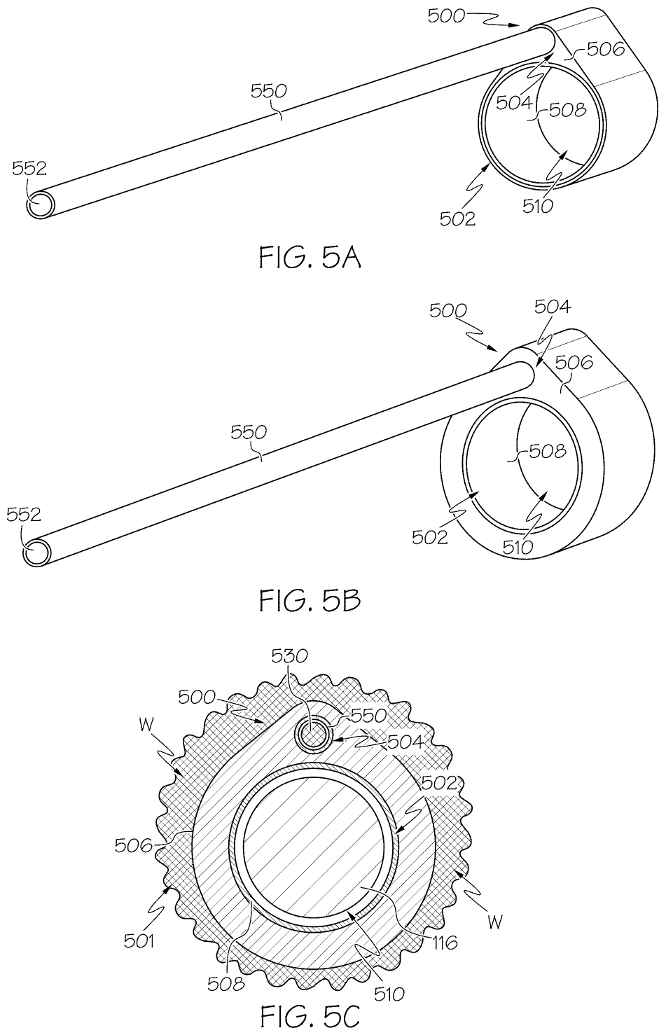

Referring now to FIGS. 5A-5C, a system 501 including another medical device 500 configured to deliver accessory devices externally to shaft 116 is shown. Medical device 500 may include a sheath 550 and an expandable member 506. Expandable member 506 may be an absorbent member, sponge, or other suitable material configured to absorb fluid or expand into a larger size, such as a pliable, porous material allowing expandable member 506 and its features to conform to tortuous body lumens or shaft 116.

FIG. 5A show expandable member 506 in a compressed state. In a compressed state, expandable member 506 may occupy a smaller volume, making it easier to insert into body lumens. FIG. 5B shows expandable member 506 in an expanded state. Upon contacting fluids present in the body or through injection of a separate fluid through, for example, an auxiliary lumen 504, expandable member 506 may expand to the expanded state shown in FIG. 5B. In the expanded state of FIG. 5B, expandable member 506 may be anchored to a body lumen, as discussed below.

Expandable member 506 may include a central lumen 502 (e.g., a primary lumen). A ring 508 may be disposed within central lumen 502. Ring 508 may have a tubular shape, with a circular cross-section and a consistent diameter along its length. Ring 508 may define a lumen 510. Ring 508 may be sized and shaped to receive shaft 116 in lumen 510. In some examples, a diameter of ring 508 may be such that shaft 116 may be longitudinally and/or rotationally movable relative to ring 508. In other examples, a diameter of ring 508 may be such that shaft 116 may not be longitudinally and/or rotationally movable relative to ring 508. Ring 508 may be sufficiently rigid such that lumen 510 has a constant diameter for receiving shaft 116 and allows a desired amount of movement of shaft 116 relative to ring 508. Ring 508 may inhibit expandable member 506 from expanding radially inward, such that expandable member 506 expands radially outward to anchor onto walls of a body lumen, as described below.

Sheath 550 may extend through a second lumen 504 (e.g., an auxiliary lumen) of expandable member 506. For example, sheath 550 may be fixedly coupled to expandable member 506. Sheath 550 may have a lumen 552 extending longitudinally through sheath 550. Lumen 552 may have any of the dimensions discussed above for auxiliary lumen 204. For example, sheath 550 may have a diameter sufficient to receive an accessory device. Lumen 504 may extend longitudinally through a portion of expandable member 506 radially outward of central lumen 502, relative to a central longitudinal axis extending through lumen 502. In an embodiment, a plurality of lumen(s) 504 may be included in medical device 500, for example, arranged circumferentially about central lumen 502.

In use, shaft 116 may be inserted into lumen 502. Shaft 116, along with device 500, may be navigated to a procedure site with expandable member 506 in a compressed state (FIG. 5A). After distal tip 120 is positioned at the procedure site, expandable member 506 may transition to the expanded state through contact with body fluid, shown in FIGS. 5B and 5C. Alternatively, expandable member 506 may transition to the expanded state by injecting a separate fluid through, for example, auxiliary lumen 504 or from a lumen of distal tip 120.

As shown in FIG. 5C, expandable member 506 may contact walls “W” of the body lumen. Thus, expandable member 506 may anchor sheath 550 in a desired location. Ring 508 may inhibit expandable member 506 from expanding radially inward and restrict movement of shaft 116 relative to expandable member 506. Subsequently, an accessory device 530 (FIG. 5C) may be inserted into a proximal opening of lumen 552 and extend out of a distal opening of lumen 552. Accessory device 530 may be outside shaft 116 (e.g., an over the scope device). For example, accessory device 530 may be a grasper, clip, forceps, hook, or other device. The user may use accessory device 530 to anchor to a target tissue. Accessory device 530 may provide traction to a target tissue. The user may use another accessory device (e.g., instrument 110) to resect or otherwise treat the target tissue. For example, medical instrument 110 may be a knife. The shaft 116 may be movable through lumen 510 to position distal tip 120 at a desired location.

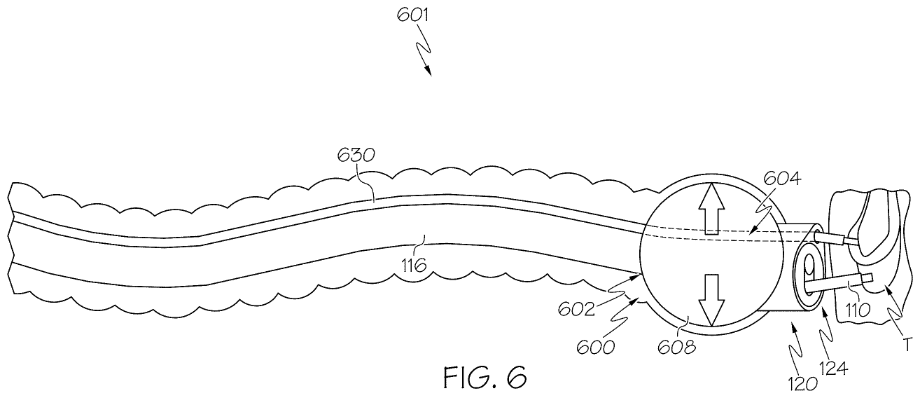

Referring now to FIG. 6, a system 601 having a sleeve 600 allowing the passage of accessory devices outside of shaft 116 (e.g., over the scope) is shown. Sleeve 600 may include a balloon 608, a central lumen 602, and an auxiliary lumen 604. In the embodiment, balloon 608 may be utilized to anchor onto a body lumen's walls, as shown in FIG. 6.

Sleeve 600 may define a central lumen 602 that is similar to any of central lumens 202, 302, 402, and 502. For example, central lumen 602 may be sized and shaped to receive shaft 116. In some examples, a ring (not shown) having any of the properties of ring 508 may be positioned between shaft 116 and balloon 608. However, any other suitable structure may be used. Shaft 116 may be movable (e.g., rotatably and/or longitudinally) through lumen 602. Auxiliary lumen 604 may be sized and shaped to receive an accessory device 630. Auxiliary lumen 604 may extend longitudinally along the length of sleeve 600, for example, as a tube or similar structure that is capable of extending through balloon 608. Auxiliary lumen 604 may extend proximally to a proximal end of shaft 116, while a remainder of sleeve 600 may be disposed only about a distal portion of shaft 116.

Balloon 608 may be an expandable member configured to transition between a collapsed configuration and an expanded configuration (e.g., the expanded configuration shown in FIG. 6). Balloon 608 may be coupled to a fluid source. In some examples, the fluid source may be integrated into or coupled to auxiliary lumen 604. Alternatively, any other fluid source known in the art may be used to provide fluid (e.g., a liquid or gas) to balloon 608.

In use, sleeve 600 may be positioned over a distal portion of a shaft. For example, sleeve 600 may be inserted into the body lumen with balloon 608 in a deflated configuration to facilitate insertion. Once positioned at the desired location within the body lumen, balloon 608 may be inflated. For example, air or saline solution through the fluid source using an inflation port or mechanism. As balloon 608 inflates, balloon 608 may expand in a radial direction, as shown by the arrows in FIG. 6, exerting outward pressure against the walls of the body lumen.

Balloon 608 may anchor auxiliary lumen 604 and any accessory devices 630 extending therethrough relative to the walls of the body lumen. For example, as shown in FIG. 6, a distal end of accessory device 630 may include a grasper, and the grasper may grasp onto a target tissue “T.” Shaft 116 may be movable relative to sleeve 600 to position shaft 116 in a desired position.

A medical instrument 110 may be inserted into a working channel of shaft 116 and extend distally out of a distal opening of the working channel. Medical instrument 110 may be used to resect the target tissue “T,” as shown in FIG. 6. For example, medical instrument 110 may be a knife. As medical instrument 110 resects the target tissue “T,” accessory device 630 may apply traction to target tissue “T” and pull target tissue “T” away from surrounding portions of the walls of the body lumen. Balloon 608 may be deflated before medical device 100 is removed from the body lumen. In some examples, accessory device 630 may continue to grasp target tissue “T” and remove target tissue “T” from the body lumen as medical device 100 is removed. In alternatives, instrument 110 may be removed from the working channel, and another device (e.g., a basket, snare, forceps, or other device) may be inserted into the working channel and used to capture the target tissue “T”.

Each of the aforementioned systems, devices, assemblies, and methods may be used to facilitate treatment of a target treatment site by allowing delivery of a plurality of medical devices. It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

1. A medical device, comprising:
a first wall defining a first lumen, wherein the first lumen is configured to receive a shaft of a medical scope;
at least one second wall defining at least one second lumen, wherein the at least one second lumen is configured to receive an accessory device; and
wherein the at least one second wall includes a perforation extending longitudinally along the at least one second wall.

2. The medical device of claim 1, wherein the medical device comprises a plurality of second walls defining a plurality of second lumens.

3. The medical device of claim 2, wherein the plurality of second lumens is positioned circumferentially around the first lumen.

4. The medical device of claim 3, wherein each of the plurality of second walls include a perforation extending longitudinally along a respective second wall of the plurality of second walls.

5. The medical device of claim 4, wherein the perforation is on an outer surface of the respective second wall.

6. The medical device of claim 5, wherein the perforation extends from a distalmost end of the respective second wall to a proximalmost end of the respective second wall.

7. The medical device of claim 4, wherein the perforation is a second perforation, and wherein the first wall includes a first perforation.

8. The medical device of claim 7, wherein the first perforation extends longitudinally along the first wall.

9. The medical device of claim 7, wherein the first perforation extends from a distalmost end of the first wall to a proximalmost end of the first wall.

10. The medical device of claim 1, wherein the medical device is a sleeve that is configured to extend from a distal tip of the shaft to a proximal portion of the shaft that remains external to a body lumen.

11. The medical device of claim 1, wherein a tab is formed adjacent to a proximal end of the at least one second wall.

12. The medical device of claim 11, wherein a second tab is formed adjacent to a proximal end of the first wall.

13. The medical device of claim 1, wherein a proximal end of the first wall has a recessed or notched portion.

14. The medical device of claim 1, wherein the at least one second lumen has an open proximalmost end and an open distalmost end and wherein the first lumen has an open proximalmost end and an open distalmost end.

15. The medical device of claim 1, wherein a membrane includes the first wall and the at least one second wall.

16. A medical device, comprising:
a membrane defining a central lumen configured to receive a scope device and a plurality of outer lumens,
wherein each of the plurality of outer lumens is configured to receive an accessory device; and
wherein a perforation extends longitudinally along a wall defining at least one of the plurality of outer lumens.

17. The medical device of claim 16, wherein the membrane includes a plurality of perforations, wherein each perforation of the plurality of perforations extends longitudinally along a respective wall defining each of the plurality of outer lumens.

18. A medical device, comprising:
an expandable member;
a first lumen extending through the expandable member and configured to receive a shaft of a scope device; and
a second lumen extending through the expandable member and configured to receive an accessory device.

19. The medical device of claim 18, wherein the expandable member includes an absorbent member, and wherein the medical device further includes a ring disposed within the first lumen.

20. The medical device of claim 18, wherein the expandable member includes a balloon.
